(19) United States
(12) Patent Application Publication
(10) Pub. No.: US 2007/0078100 A1
(43) Pub. Date: Apr. 5, 2007

(54) Methods of detecting poly(adp-ribose) polymerase and other nad+ utilizing enzymes

(75) Inventors: Paul J. Hergenrother, Champaign, IL (US); Karson S. Putt, Urbana, IL (US)

Correspondence Address: EVAN LAW GROUP LLC, 600 WEST JACKSON BLVD., SUITE 625, CHICAGO, IL, 60661, US

(73) Assignee: THE BOARD OF TRUSTEES OF THE UNIVERSITY OF ILLINOI, URBANA, IL (US)

(21) Appl. No.: 10/595,360

(22) PCT Filed: Oct. 14, 2004
(86) PCT No.: PCT/US04/34010

§ 371 Date: Dec. 21, 2006

Related U.S. Application Data
(60) Provisional application No. 60/510,916, filed on Oct. 14, 2003.

(51) Int. Cl. C12Q 1/26; C07H 19/207

(52) U.S. Cl. 514/43; 536/26.24; 435/25

(57) ABSTRACT

Methods for detecting poly (ADP-ribose) polymerase and other NAD+ utilizing enzymes.

### CROSS REFERENCE TO RELATED APPLICATION

This application claims the benefit of U.S. Provisional Application No. 60/510,916 filed 14 Oct. 2003.

### FEDERALLY SPONSORED RESEARCH OR DEVELOPMENT

The subject matter of this application may in part have been funded by the National Science Foundation (NSF) Grant No. 0134779. The government may have certain rights in this invention.

### BACKGROUND

The mammalian cell possesses elaborate systems that regulate both life and death. Upon insult through chemical, biological, or other means, the cell activates multiple mechanisms in an attempt to survive. For example, in response to DNA damage the enzyme poly(ADP-ribose) polymerase (PARP) binds to DNA and uses the ADP-ribose moiety of NAD as a substrate to poly(ADP-ribosylate) a variety of proteins on glutamate residues (FIG. 1) [1,2]. The ADP-ribosyl units are added as both linear and branched chains, and more than 100 monomers can be appended in forming the poly(ADP-ribose) polymer. The formation of these polymers dramatically alters the properties of the acceptor proteins, and this modification initiates the DNA damage control and repair process. The modification of PARP with the ribosyl polymer eventually leads to dissociation of the enzyme from the DNA. This poly(ADP-ribosylation) is transient, as the modified proteins are rapidly restored to their original states by a poly(ADP-ribose) glycohydrolase (PARG) [3]. The DNA repair role for PARP in response to alkylating agents and ionizing radiation is supported by studies in PARP deficient cell lines [4] and organisms [5]. Therefore, PARP enzymatic activity appears to have a cytoprotective role within the cell, and inhibition of PARP with small molecules is known to increase the sensitivity of cells to cytotoxic agents [6-8].

However, it is also apparent that PARP enzymatic activity is a significant contributor to necrotic cell death (see FIG. 2). PARP activity leads to the depletion of NAD+ and ATP energy stores inside the cell, ultimately leading to necrosis [9-11]. PARP activity therefore has a cytotoxic role within the cell, and inhibitors of PARP have been shown to prevent necrotic cell death in a wide variety of in vivo models of ischemia and reactive oxygen species-induced injury [10-13].

These contrasting and seemingly paradoxical roles for PARP have made it the subject of considerable biochemical and medicinal interest. Although at least six members of the PARP family of enzymes have been identified, PARP-1, a 113 kDa nuclear enzyme, was the first PARP discovered and remains the most well characterized member of the family. During apoptosis, the endopeptidase caspase-3 cleaves PARP-1 after a DEVD amino acid sequence to yield p89 and p24 fragments. This cleavage separates the DNA binding domain of PARP-1 from its catalytic domain, thereby rendering the enzyme catalytically inactive [14-17]. This cleavage activates futile cycles of DNA damage and repair, thereby depleting the cellular ATP/NAD+ pool that leads to cell death. The other five members of the PARP family (PARP-2, PARP-3, VPARP, tankyrase I, and tankyrase II) have varying degrees of sequence homology with PARP-1, but their in vivo functions remain poorly understood [18].

Three-dimensional X-ray crystallographic structures of the catalytic domain of PARP-1 have been solved in the presence of small molecule inhibitors, which appear to occupy the nicotinamide binding site of the enzyme [19,20]. Because PARP inhibitors have been shown to both enhance the effects of cytotoxic agents and avert necrotic cell death, these compounds have been suggested as therapeutic agents for treating disease states ranging from cancers to degenerative disorders [8,21-23].

Various screens have identified small molecules that inhibit PARP with reasonable potencies [24]. The standard assay for monitoring PARP activity involves the use of radiolabeled NAD+[13,24,25], although an assay that uses an antibody to ADP-ribose [26] and two recently described assays using biotinylated-NAD+ have been devleloped [27,28]. Unfortunately, the use of radioactive and/or specialized reagents (such as the aforementioned antibody and biotinylated-NAD+ reagents) in these assays can make their costs prohibitive when screening large compound collections for PARP inhibition. In addition, these assays often involve either the separation of ADP-ribose polymer product from the NAD+ substrate, or the addition of specialized streptavidin-conjugated scintillation proximity assay beads.

The present invention relates to an inexpensive, convenient and sensitive method for detecting PARP and other NAD+ utilizing enzyme activities as well as a rapid method for identifying inhibitors of these enzymes from large compound collections.

### SUMMARY

In a first aspect, the present invention is a product of the structure of compound 1.

In a second aspect, the present invention is a method of preparing compound 1 in a solution, comprising: mixing a solution containing NAD+ with alkali and acetophenone to yield an alkaline mixture; neutralizing the alkaline mixture with an acid solution to yield a neutralized mixture; and heating the neutralized mixture.

In a third aspect, the present invention is a method of detecting NAD+ in a solution, comprising converting NAD+ to a fluorescent derivative and measuring the fluorescence of the solution.

In a fourth aspect, the present invention is a method of detecting an NAD+ utilizing enzyme in solution, comprising: incubating the NAD+ utilizing enzyme with NAD+ and its substrate in a solution; converting NAD+ to a fluorescent derivative; and measuring the fluorescence of the solution, wherein a decrease in the fluorescence of the fluorescent derivative over time indicates consumption of NAD+ by the NAD+ utilizing enzyme.

In a fifth aspect, the present invention is a method of detecting an inhibitor to an NAD+ utilizing enzyme, comprising: incubating the NAD+ utilizing enzyme with NAD+ and other substrates in a solution that contains or lacks a compound; converting NAD+ to a fluorescent derivative; and measuring the fluorescence of the solution, wherein an increase in fluorescence in the solution containing the compound relative to the fluorescence in the solution lacking the compound indicates that the compound is an inhibitor to the NAD+ utilizing enzyme.

In a sixth aspect, the present invention is a method of detecting a genetic deficiency in an NAD+ utilizing enzyme, comprising: obtaining a biopsy sample from a patient; preparing a cellular lysate from the biopsy sample; performing an NAD+ utilizing enzyme activity assay on the cellular lysate, wherein the NAD+ utilizing enzyme is incubated in the presence of a substrate specific for the NAD+ utilizing enzyme and NAD+; converting NAD+ to a fluorescent derivative; and measuring the fluorescence, wherein an increase in fluorescence in the cellular lysate from the biopsy sample relative to the fluorescence in the cellular lysate from a control sample indicates the presence of a genetic deficiency in the NAD+ utilizing enzyme in the biopsy sample.

In a seventh aspect, the present invention is a kit for detecting an NAD+ utilizing enzyme, comprising: NAD+; alkali; acetophenone; alcohol; acid; a control solution of compound 1; a control NAD+ utilizing enzyme reaction mixture components; and instructions.

### BRIEF DESCRIPTION OF THE FIGURES

FIG. 1 depicts the reaction catalyzed by PARP, wherein the ADP-ribose moiety of NAD+ is covalently attached to the carboxylate residues of target proteins;

FIG. 2 depicts the dual nature of PARP, wherein PARP activation can lead to DNA repair and recovery of normal cellular function or wherein it can cause energy deprivation and cell death;

FIG. 3 depicts the quantitation of NAD+ by its chemical conversion into the highly fluorescent compound 1 upon reaction with acetophenone;

FIG. 4 depicts the fluorescence excitation (closed squares) and emission (open circles) spectra of compound 1;

FIG. 5 depicts the mass spectral data, wherein the parent ion peak at 764.68 amu was assigned to compound 1 and the ion peaks above 1500 may be a dimer of compound 1 with sodium/potassium;

FIG. 6 depicts a typical NAD+ calibration curve using the chemical conversion assay;

FIG. 7 depicts the screening of compounds for PARP-1 inhibition in a 384-well plate, wherein cells 4F, 6D, 6G, and 11C correspond to known PARP-1 inhibitors: 6(5H)-phenanthridinone, 4-Amino-1,8-naphthalimide, DPQ, and benzamide, respectively; and

FIG. 8 depicts use of the NAD+ quantitation assay used to evaluate the PARP-1 inhibitors: benzamide (closed diamonds), 6(5H)-phenathridinone (open squares), 4-amino-1,8-naphthalimide (closed circles), and DPQ (open triangles).

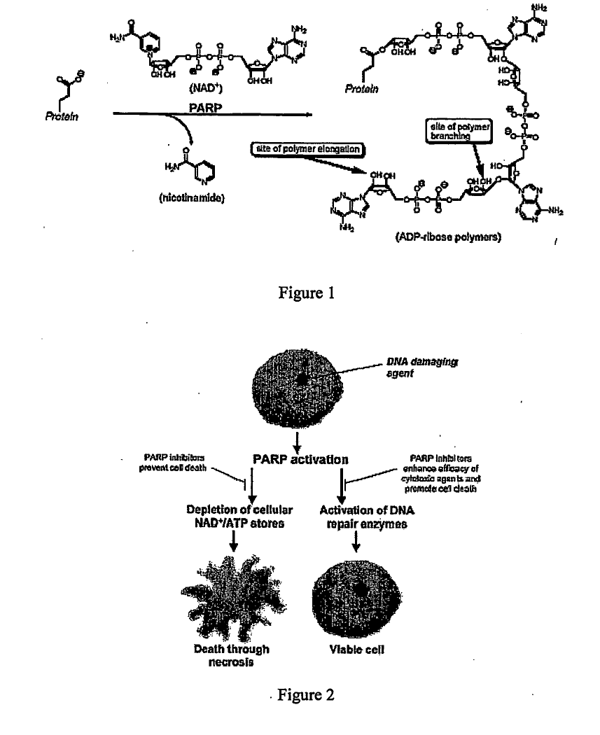

### DETAILED DESCRIPTION

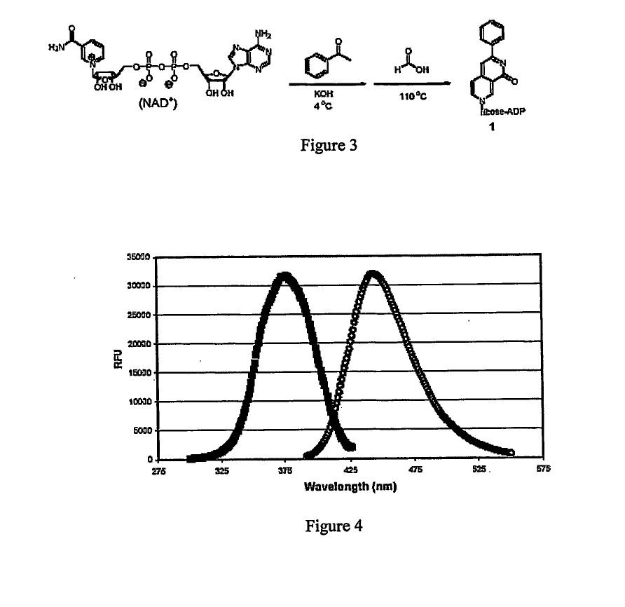

The present invention makes use of the discovery that PARP enzymes can be readily detected using a highly sensitive, convenient, simple assay that relies upon the chemical conversion of NAD+ into a highly fluorescent derivative 1 (FIG. 3). The methods of the invention may be used for the identification and evaluation of inhibitors of this entire medicinally important class of enzymes. Furthermore, the methods of the invention are broadly amenable for the detection of other NAD+ utilizing enzyme activities as well as identification of therapeutic compounds useful for the treatment of diseases that are caused by enzymes that use NAD+ as a substrate.

The following is presented to aid the practitioner, although other methods, techniques, cells, reagents, and approaches can be used.

To develop a non-radiometric method of measuring PARP activity, protocols were developed in which the reaction by-product (nicotinamide) or the starting material (NAD+) could be conveniently quantitated by either a UV/visible or spectrofluorometric measurements. It is well recognized that ideal enzyme assays enable one to directly measure the amount of the products generated for a given enzymatic transformation; however, multiple enzyme assays have been developed for systems in which product formation can neither be conveniently nor directly monitored. In these cases, it is possible to assess enzymatic activity based on the amount of substrate remaining after the enzymatic reaction. Thus, in the case of the reaction catalyzed by PARP, quantitation of the NAD+ remaining after the PARP-catalyzed reaction appeared as an attractive alternative to the standard PARP assay in which activity is determined by the amount of radioactivity transferred to PARP through the use of 3H-NAD+.

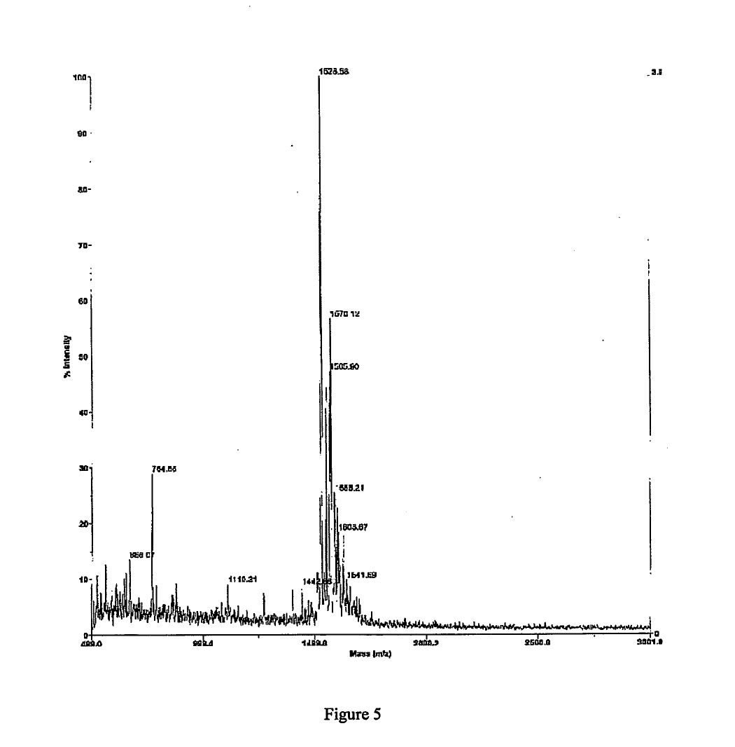

It is known that N-alkylpyridinium compounds can be converted to fluorescent molecules through reaction with a ketone followed by heating in excess acid [29]. These methods have been developed largely to quantitate N-methylnicotinamide (NMN), a nicotinic acid metabolite [30-33]. More recently, an optimized protocol for NMN determination has been reported using acetophenone as the ketone and acidifying with formic acid [34-36]. To adapt this method to the determination of PARP enzymatic activity, the amount of NAD+ remaining after the PARP reaction was measured by treating the reaction mixture with acetophenone in base, followed by incubation at 110° C. with formic acid (FIG. 3). It was discovered that the product of this reaction has a strong fluorescence emission at 444 nm. The excitation and emission spectra of this product are shown in FIG. 4. The structure of this product was assigned as compound 1 (FIG. 3), based on analogy with other reactions of N-alkylpyridines with ketones, and taking into account mass spectral data that were obtained (764.68 amu; FIG. 5).

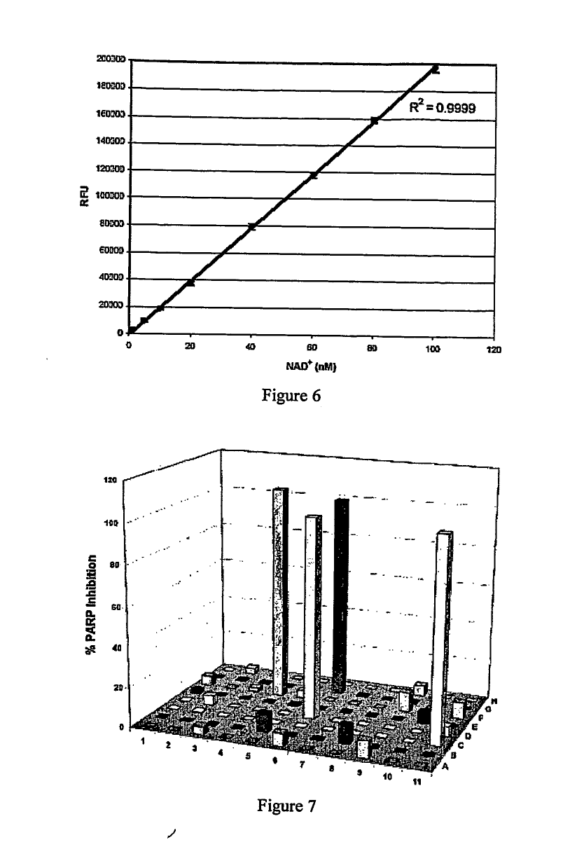

A calibration curve was produced for the reaction in FIG. 3 using various amounts of NAD+. As shown in FIG. 6, this method is highly sensitive and can detect NAD+ solutions as low as 10 pM, and the fluorescence is linear over the range of 1 nM -100 μM. Importantly, control studies revealed that nicotinamide (the by-product in the PARP reaction) does not react under the assay conditions, and NAD+ and nicotinamide have no significant intrinsic fluorescence emission at 444 nm.

As there is substantial interest in the development of small molecule inhibitors of the PARP enzymes, the chemical quantitation of NAD+ was applied to PARP in the context of inhibitor evaluation. Specifically, a new method was developed to screen large collections of compounds for inhibition of the activity of the PARP family of enzymes. Thus, 88 compounds were placed into the individual wells of a 384-well plate, with each compound assessed in quadruplicate. Four of these 88 compounds were known PARP inhibitors, whereas the other 84 compounds were from a small in-house collection of small molecules. To assess the sensitivity of the assay in this high-throughput application, the four PARP inhibitors ranged from highly potent (DPQ, 4-amino-1,8-napthalimide), to less potent (6(5H)-phenanthridinone, benzamide). The structures of these commercially available PARP inhibitors and their reported literature IC₅₀ values are provided in Table 1.

TABLE 1

| | | | literature |
|---|---|---|---|
| name | structure | IC₅₀ value | IC₅₀ value |
| DPQ | 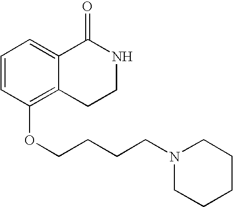 | 37 nM | 40 nM [37]; 800 nM [40]1000 nM [38]; 3500 nM [41] |
| 4-amino-1,6-naphthalimide | 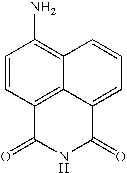 | 153 nM | 180 nM [39] |
| 6(5H)-phenanthridinons | 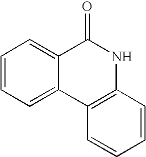 | 305 nM | 300 nM [39] |
| benzamide | 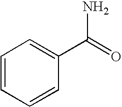 | 1444 nM | 1300 nM [28]; 22 μM [39] |

The compounds were delivered into the wells of the 384-well plate using a high-throughput pin transfer device, and the compounds were tested at a final concentration of 10 μM. The use of the pin transfer device and moderate compound concentrations are the precise conditions that would be used in a high-throughput screen of a large collection of compounds. As each compound was present in quadruplicate on the plate, two of the wells were used to evaluate any intrinsic fluorescence of the compounds (no PARP added), and the other two wells were used in the quantitation of NAD+. After addition of PARP-1 and nicked DNA, the reaction was allowed to proceed for 20 minutes at room temperature. At this point acetophenone/KOH was added, which serves to both quench the PARP-1 enzymatic activity and to begin the conversion of any remaining NAD+ to the fluorophore. After 10 min at 4° C. in the presence of acetophenone/KOH, a solution of formic acid was added and the mixture was incubated in a 110° C. oven for 5 minutes. The fluorophore thus produced was found to be stable in the dark at room temperature for 2 hours. The results of this experiment are shown in FIG. 7. As indicated by the graph, the four known inhibitors are readily apparent as hits in the assay, and none of the other compounds gave a significant signal.

This assay was also applied to determine the IC₅₀ values of the known PARP inhibitors. For these determinations, the inhibitors, at a variety of concentrations, were added to a 96-well plate containing NAD+ and nicked DNA. PARP-1 was then added, and the reaction was allowed to proceed for 15 min. At this point acetophenone/KOH was added, which serves to both quench the PARP-1 enzymatic activity and to begin the conversion of any remaining NAD+ to the fluorophore. After 10 min at 4° C. in the presence of acetophenone/KOH, a solution of formic acid was added and the mixture was incubated in a 110° C. oven for 5 minutes. The fluorophore thus produced was found to be stable in the dark at room temperature for 2 hours.

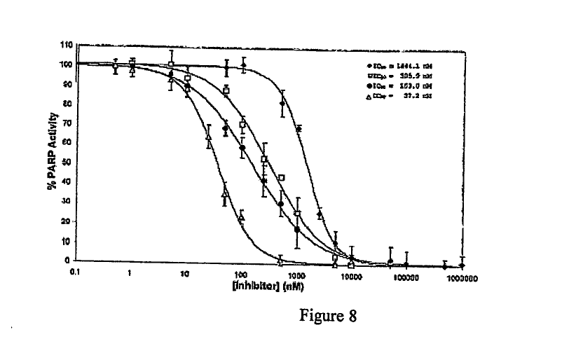

The results of the assay in the presence of the four PARP-1 inhibitors are shown in FIG. 8, and the IC₅₀'s in comparison to the reported literature values are listed in Table 1. Excellent correlation was obtained between this NAD+ quantitation method and the literature values [28,37-39]. Putative inhibitors that contain N-alkyl pyridinium moieties would give a large background in this assay; thus, these compounds should be evaluated via another method. However, such compounds are typically not potent PARP inhibitors [22].

To ensure that the initial rate remained constant in the IC₅₀ determination assay, the PARP reactions were only allowed to proceed to 8-15% completion. Even at this low level of conversion, the signal-to-noise level was more than adequate, and the assay was found to be highly reproducible; each data point in FIG. 8 was determined in quadruplicate, and the error bars are shown on the graph. In addition, only very low levels of NAD+ were used in this assay (100 nM). This low concentration, coupled with the minimal volume required by the 96- or 384-well format, allowed extremely small quantities of PARP to be used. Although PARP-1 is commercially available, it is quite expensive, and thus it is very useful to minimize the quantity of PARP-1 consumed, especially if large compound collections are to be screened or multiple IC₅₀ values are to be determined. However, NAD+ concentrations up to 100 μM can also be used, if one desires a direct correlation with other assay methods that typically use micromolar amounts of NAD+ substrate.

The methods contemplated by the present invention may be readily extended to detecting other enzyme activities that use NAD+ as a substrate. In this regard, one only needs to modify the procedure to substitute the co-substrate required for the particular enzyme of interest. For example, aldehyde dehydrogenase activities may be detected in mixtures that contain aldehyde and NAD+. The presence of the NAD+ utilizing activity would be revealed by a reduction in the production of compound 1 over time, since the enzyme will use NAD+ as a substrate during the reaction.

Furthermore, the methods disclosed herein should be amenable to detecting a genetic deficiency in enzymes that use NAD+, which may serve as a diagnostic indicator of genetic disease states. The phrase “genetic deficiency” refers to any altered expression levels of an otherwise wild-type functioning enzyme relative to that found in the general population or to any genetic variation within the coding sequence of an otherwise wild-type expressed enzyme that results in altered enzyme function relative to that found in the general population. Thus, a genetic deficiency will result in aberrant enzyme activity levels in a typical enzyme activity assay, relative to that observed for the enzyme found in the general population.

For example, defects in the activity of long-chain 3-hydroxyacyl-CoA dehydrogenase, an enzyme which catalyzes the conversion of 3-hydroxyacyl-CoA in the presence of NAD+ to yield 3-oxoacyl-CoA and NADH, may reveal a β-oxidation defect and a propensity to develop hypoglycemia or cardiomyopathy. Allelic variations in the genetic coding sequence encoding the α subunit of the enzyme result in reduced functional activity for the enzyme. One may use the prese invention to identify patients with this deficiency by analyzing patient biopsy samples for the enzyme's activity relative to control samples taken from the general population. Biopsy samples with reduced activity for this NAD+ utilizing enzyme relative to control samples would be expected to consume less NAD+ in an enzyme activity assay and produce greater fluorescence following the chemical conversion of NAD+ to compound 1 relative to the control samples. In this fashion, the present invention has utility as a diagnostic tool in the clinical realm.

The methods contemplated by the invention can also be used to rapidly screen large compound catalogs to identify and characterize inhibitors of NAD+ utilizing enzymes. Similar to that described for the screening of PARP inhibitors, the methods disclosed herein should be amenable to the identification of inhibitors of other NAD+ utilizing enzyme targets that have a role in diseases. From this standpoint, the described methods may be used to evaluate the efficacy of disease treatment procedures that rely upon inhibiting NAD+ utilizing enzyme activities or for which NAD+ utilizing enzyme activities serve as a marker of the disease state. For example, a biopsy sample may be acquired and the effectiveness of an inhibitor for a given NAD+ utilizing enzyme activity may be evaluated using the invention. The extreme sensitivity of the methods, when combined with high-throughput screening approaches, should enable identification of potential inhibitors for NAD+ utilizing enzymes for a given biopsy sample in an allele-specific manner. Thus, the selection of a therapeutic drug may be individually tailored to the type of disorder and patient to be treated.

Methods for detecting PARP and other NAD+ utilizing enzyme activities can be included in a kit, container, pack, or dispenser together with instructions for use. Preferred kits may include NAD+ solution, 88% formic acid, 2 M KOH solution, 20% acetophenone (in EtOH) solution, a suitable enzyme assay buffer, and suitable control reagents, such as a solution of compound 1, PARP and DNA. When the invention is supplied as a kit, the different components of the composition may be packaged in separate containers and admixed immediately before use. Such packaging of the components separately may permit better long-term storage.

The reagents included in the kits can be supplied in containers of any sort such that the life of the different components are preserved and are not adsorbed or altered by the materials of the container. For example, sealed glass ampules may contain buffer that have been packaged under a neutral non-reacting gas, such as nitrogen. Ampules may consist of any suitable material, such as glass, organic polymers, such as polycarbonate, polystyrene, etc., ceramic, metal or any other material typically employed to hold reagents. Other examples of suitable containers include bottles that may be fabricated from similar substances as ampules, and envelopes, that may consist of foil-lined interiors, such as aluminum or an alloy. Other containers include test tubes, vials, flasks, bottles, syringes, etc. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

Kits may also be supplied with instructional materials. Instructions may be printed on paper or other substrate, and/or may be supplied as an electronic-readable medium, such as a floppy disc, CD-ROM, DVD-ROM, Zip disc, videotape, audio tape, etc. Detailed instructions may not be physically associated with the kit; instead, a user may be directed to an internet web site specified by the manufacturer or distributor of the kit, or supplied as electronic mail.

### EXAMPLES

### Example 1

### Chemical Conversion of NAD+ to a Highly Fluorescent Derivative

Tigh specific activity PARP-1 and activated DNA were purchased from Trevigen, (Gaithersburg, Md.). Acetophenone, benzamide, 6(5H)-phenanthridinone and 3,4-dihydro-5-[4-(1-piperidinyl)butoxy]-1 (2B)-isoquinolinone (DPQ) were purchased from Sigma-Aldrich (St. Louis, Mo.). 4-12-Amno-1,8-naphthalimide was purchased from Calbiochem (San Diego, Calif.). Ninety-six well fluorescence plates, 96 well UV-visible transparent plates, 88% formic acid and all other reagents were purchased from Fisher (Chicago, Ill.). PARP assay buffer consisted of 50 mM Tris, 2 mM MgCl₂ at pH 8.0. The solutions of aqueous 2 M KOH and 20% acetophenone (in EtOH) were stable for at least 1 month at room temperature in the dark. 50 mM stock solutions of 6(5H)-Phenanthridinone, 4-Amino-1,8-naphthalimide and DPQ were prepared in DMSO. A 5 mM stock solution of benzamide was prepared in the PARP assay buffer.

Fifty microliters of 1 to 100 nM (for fluorescence) or 1 to 100 μM (for absorbance) NAD+ solutions in PARP assay buffer were added in quadruplicate to the wells of a Nunc 96-well round bottom fluorescence plate, followed by the addition of 20 μL of an aqueous 2 M KOH solution and 20 μL of a 20% acetophenone (in EtOH) solution. Higher concentrations of acetophenone can be used to increase the fluorescent signal, however most plastic microtiter plates will dissolve if higher concentrations are used. The plate was then incubated at 4° C. for 10 min. Ninety microliters of 88% formic acid was then added and the plate was incubated in an oven set at 110° C. for 5 min. The plate was allowed to cool and then read on a Criterion Analyst AD (Molecular Devices, Sunnyvale, Calif.) with an excitation of 360 nm and an emission of 445 nm (see exact settings, below). To quantitate NAD+ via absorbance, the reaction mixture was transferred from the fluorescence plate into a Falcon UV-VIS transparent 96-well plate and read on a SpectraMax Plus (Molecular Devices, Sunnyvale, Calif.) at 378 nm. The above reaction cannot be carried out directly in the UV-VIS transparent plate because the plate is not resistant to heating.

Fluorescence was measured on a Criterion Analyst AD using a 360+/−15 nm excitation filter, a 445+/−15 nm emission filter and a 400 nm cutoff dichroic mirror. The fluorophore was excited using a 1000 W continuous lamp for 1.6×10⁶ μs with 5 reads performed per well.

### Example 2

### Rapid Screening of Inhibitors to PARP-1 Enzyme Activity

Stock solutions of 88 compounds were prepared, each at a concentration of 1.25 mM in DMSO. Contained within this compound collection were the known PARP inhibitors: 4-amino-1,8-naphthalimide, benzamide, DPQ, and 6(5H)-phenanthridinone. Fifty microliters of each of these 88 stock solutions were placed into the wells of a 96-well plate (the parent plate). To test the library for PARP inhibition, 20 μL of NAD+ (at a concentration of 1.25 μM in PARP assay buffer) was added to the wells of a Costar flat bottom 384-well fluorescent plate. Subsequently, 0.2 μL of the test compounds were transferred from the parent plate into the experimental plate using a pin transfer apparatus (V & P Scientific, San Diego Calif.). To initiate the reactions, 5 μL of a solution containing both PARP (at 12.5 μg/mL) and nicked DNA (at 75 μg/mL) in PARP assay buffer were added, bringing the final concentration of PARP to 2.5 μg/mL, DNA to 15 μg/mL, NAD+ to 1 μM, and compound to 10 μM. The plate was incubated at room temperature for 20 minutes and the amount of NAD+ present was then determined by the addition of 10 μL of an aqueous 2 M KOH solution and 10 μL of a 20% acetophenone (in EtOH) solution. The plate was then incubated at 4 ° C. for 10 minutes. Forty-five microliters of 88% formic acid was then added and the plate was incubated in an oven set at 110 ° C. for 5 minutes. The plate was allowed to cool and then read on a Criterion Analyst AD (Molecular Devices, Sunnyvale, Calif.) with an excitation of 360 nm and an emission of 445 nm. Within the experimental plate, this assay was performed in duplicate.

To control for any potential fluorescence inherent in the compounds under evaluation, wells containing only the compound (at 10 μM) and NAD+ (1 μM) in PARP assay buffer (total volume of 25 μL) were analyzed alongside the experimental samples, in duplicate, within the same 384-well plate. The value of any intrinsic fluorescence detected in the compounds was subtracted out during the final analysis (see below).

Other control wells were also analyzed; these contained either 1) NAD+ with 0.2 μL of DMSO transferred into them or 2) NAD+ and PARP with 0.2 μL of DMSO transferred into them. The amount of PARP inhibition was determined by first subtracting out any intrinsic fluorescence of the test compounds. Next, the average value of the control wells containing only NAD+ were set as 100% inhibition, while the control wells containing NAD+ and PARP were set as 0% inhibition. Lastly, the values of the test compounds were converted to a percentage of PARP inhibition and plotted.

### Example 3

### Determination of IC₅₀ Values for PARP Inhibitors

To determine IC₅₀ values of the PARP inhibitors, 20 μL of a 250 nM solution of NAD+ in PARP assay buffer, 10 μL of activated DNA at a concentration of 50 μg/mL (in PARP assay buffer) and 10 μL of the inhibitors at varying concentrations (in PARP assay buffer) were added into the wells of a 96-well plate. The reaction was initiated by adding 10 μL of PARP at a concentration of 10 μg/mL (in PARP assay buffer), bringing the final concentration to 2 μg/mL PARP, 10 μg/mL DNA, 100 nM NAD+ with varying concentrations of inhibitors in a total volume of 50 μL. The plate was incubated for 15 min at room temperature and the amount of NAD+ was then determined by the fluorescence method as described above for the calibration curve. The average value of control wells containing only NAD+ was set as 0% PARP activity, while the average value of control wells containing NAD+ and PARP (but no inhibitor) was set as 100% PARP activity. Any intrinsic fluorescence exhibited by the PARP inhibitors was subtracted out and the values obtained from the various concentrations of inhibitors were converted to a percentage of PARP activity and plotted. All data points in FIG. 8 were determined in quadruplicate.

Graphs were analyzed using Table Curve 2D. NAD+ standard curves were fitted with a least squares linear model and inhibitor curves were fitted with a logistic dose response curve.

### REFERENCES

1. Tong, W.-M.; Cortes, U.; Wang, Z.-Q., Poly(ADP-ribose) polymerase: a guardian angel protecting the genome and suppressing tumorigenesis, Biochim. Biophys. Acta 1552 (2001) 27-37.

2. Burkle, A., PARP-1: A regulator of genomic stability linked with mammalian longevity, Chembiochem 2 (2001) 725-728.

3. Davidovic, L.; Vodenicharov, M.; Affar, E. B.; Poirier, G. G., Importance of poly(ADP-ribose) glycohydrolase in the control of poly(ADP-ribose) metabolism, Exp. Cell Res. 268 (2001) 7-13.

4. Le Page, F.; Schreiber, V.; Dherin, C.; de Murcia, G.; Boiteux, S., Poly(ADP-ribose) polymerase-1(PARP-1) is required in Murine cell lines for base excision repair of oxidative DNA damage in the absence of DNA polymerase β, J. Biol. Chem. 278 (2003) 18471-18477.

5. Shall, S.; de Murcia, G., Poly(ADP-ribose) polymerase-1: what have we learned from the deficient mouse model, Mutation Res. 460 (2000) 1-15.

6. Delaney, C. A.; Wang, L.-Z.; Kyle, S.; White, A. W.; Calvert, A. H.; Curtin, N. J.; Durkacz, B. W.; Hostomsky, Z.; Newell, D. R., Potentiation of temozolomide and topotecan growth inhibition and cytotoxicity by novel poly(adenosine diphosphoribose) polymerase inhibitors in a panel of human tumor cell lines, Clin. Cancer Res. 6 (2000) 2860-2867.

7. Griffin, R. J.; Curtin, N. J.; Newell, D. R.; Golding, B. T.; Durkacz, B. W.; Calvert, A. H., The role of inhibitors of poly(ADP-ribose) polymerase as resistance-modifying agents in cancer therapy, Biochimie 77 (1995) 408-422.

8. Cosi, C., New inhibitors of poly(ADP-ribose) polymerase and their potential therapeutic targets, Expert Opin. Ther. Patents 12 (2002) 1047-1070.

9. Sims, J. L.; Berger, S. J.; Berger, N. A., Poly (ADP-ribose) polymerase inhibitors preserve nicotinamide adenine dinucleotide and adensosine 5′-triphosphate polls in DNA-Damaged cells: Mechanism of stimulation of unscheduled DNA synthesis, Biochemistry 22 (1983) 5188-5194.

10. Schraufstatter, I. U.; Hyslop, P. A.; Hinshaw, D. B.; Spragg, R. G.; Sklar, L. A.; Cochrane, C. G., Hydrogen peroxide-induced injury of cells and its prevention by inhibitors of poly(ADP-ribose) polymerase, Proc. Natl. Acad. Sci. 83 (1986) 4908-4912.

11. Szabo, C.; Dawson, V. L., Roly of poly(ADP-ribose) sythetase in inflammation and ischaemia-reperfusion, Trends Pharm. Sci. 19 (1998) 287-298.

12. Thiemermann, C.; Bowes, J.; Myint, F. P.; Vane, J. R., Inhibition of the activity of poly(ADP ribose) synthetase reduces ischemia-reperfuion injury in the heart and skeletal muscle, Proc. Natl. Acad. Sci. 94 (1997) 679-683.

13. Zhang, J.; Lautar, S.; Huang, S.; Ramsey, C.; Cheung, A.; Li, J.-H., GP16150 prevents H2O2 cytotoxicity by inhibiting poly(ADP-ribose) polymerase, Biochem. Biophys. Res. Comm. 278 (2000) 590-598.

14. Kaufmann, S. H.; Desnoyers, S.; Ottaviano, Y.; Davidson, N. E.; Poirier, G. G., Specific proteolytic cleavage of poly(ADP-ribose) polymerase: an early marker of chemotherapy-induced apoptosis, Cancer Res. 53 (1993) 3976-3985.

15. Lazenik, Y. A.; Kaufinann, S. H.; Desnoyers, S.; Poirier, G. G.; Earnshaw, W. C., Cleavage of poly(ADP-ribose) polymerase by a proteinase with properties like ICE, Nature 371 (1994) 346-347.

16. Tewari, M.; Quan, L. T.; O'Rourke, K.; Desnoyers, S.; Zeng, Z.; Beidler, D. R.; Poirier, G. G.; S., S. G.; Dixit, V. M., Yama/CPP32b, a mammalian homolog of CED-3 is a CrmA-inhibitable protease that cleaves the death substrate poly(ADP-ribose) polymerase, Cell 81 (1995) 801-809.

17. Scovassi, A. I.; Poirier, G. G., Poly(ADP-ribosylation) and apoptosis, Mol. Cell. Biochem. 199 (1999) 125-137.

18. Smith, S., The world according to PARP, Trends Biochem. Sci. 26 (2001) 174-179.

19. Ruf, A.; de Murcia, J. M.; de Murcia, G. M.; Schulz, G., Structure of the catalytic fragment of poly(ADP-ribose) polymerase from chicken, Proc. Natl. Acad. Sci. 93 (1996) 7481-7485.

20. Ruf, A.; de Murcia, G.; Schulz, G., Inhibitor and NAD+ binding to poly(ADP-ribose) polymerase as derived from crystal structures and homology modeling, Biochemistry 37 (1998) 3893-3900.

21. Virag, L.; Szabo, C., The therapeutic potential of poly(ADP-ribose) polymerase inhibitors, Pharnacol. Rev. 54 (2002) 375-429.

22. Southan, G. J.; Szabo, C., Poly(ADP-ribose) polymerase inhibitors, Curr. Med. Chem. 10 (2003) 321-340.

23. Tentori, L.; Portarena, I.; Graziani, G., Potential clinical applications of poly(ADP-ribose) polymerase (PARP) inhibitors, Pharmacol. Res. 45 (2002) 73-85.

24. Li, J.-H.; Serdyuk, L.; Ferraris, D. V.; Xiao, G.; Tays, K. L.; Kletzly, P. W.; Li, W.; Lautar, S.; Zhang, J.; Kalish, V. J., Synthesis of substituted 5[H]phenanthridin-6-ones as potent poly(ADP-ribose) polymerase-1 (PARP1) inhibitors, Bioorg. Med. Chem. Lett. 11 (2001) 1687-1690.

25. Dillon, K. J.; Smith, G. C. M.; Martin, N. M. B., A FlashPlate Assay for the Identification of PARP-1 Inhibitors, J. Biomol. Screening 8 (2003) 347-352.

26. Decker, P.; Miranda, E. A.; de Murcia, G.; Muller, S., An improved nonisotopic test to screen a large series of new inhibitor molecules of poly(ADP-ribose) polymerase activity for therapeutic applications, Clin. Cancer Res. 5 (1999) 1169-1172.

27. Brown, J. A.; Marala, R. B., Development of a high-throughput screening-amenable assay for human poly(ADP-ribose) polymerase inhibitors, J. Pharma. Toxicol. Meth. 47 (2002) 137-141.

28. Cheung, A.; Zhang, J., A scintillation proximity assay for poly(ADP-ribose) polymerase, Anal. Biochem. 282 (2000) 24-28.

29. Huff, J. W., The fluorescent condensation product of N-methylnicotinamide and acetone, J. Biol. Chem. 167 (1947) 151-156.

30. Huff, J. W.; Perlzweig, W. A., The fluorescent condensation product of N-methylnicotinamide and acetone, J. Biol. Chem. 167 (1947) 157-167.

31. Levitas, N.; Robinson, J.; Rosen, F.; Huff, J. W.; Perlzweig, W. A., The fluorescent condensation product of N-methylnicotinamide and acetone, J. Biol. Chem. 167 (1947) 169-175.

32. Robinson, J.; Levitas, N.; Rosen, F.; Perlzweig, W. A., The fluorescent condensation product of N-methylnicotinamide and acetone, J. Biol. Chem. 170 (1947) 653-659.

33. Carpenter, K. J.; Kodicek, E., The fluorimetric estimation of N-methylnicotinamide and its differentiation from coenzyme I, Biochem. J. 46 (1950) 421-426.

34. Clark, B. R.; Halpern, R. M.; Smith, R. A., A fluorimetric method for quantitation in the picomole range of N-methylnicotinamide and nicotinamide in serum, Anal. Biochem. 68 (1975) 54-61.

35. Nakamura, H.; Tamura, Z., Fluorometric assay of α-methylene carbonyl compounds with N-methylnicotinamide chloride, Anal. Chem. 50 (1978) 2047-2051.

36. Sano, A.; Nakamura, H., Fluorometric determination of N-nitroso-N-methylurea with nicotinamide and acetophenone, Anal. Sci. 17 (2001) 375-378.

37. Eliasson, M. J. L.; Sampei, K.; Mandir, A. S.; Hurn, P. D.; Traystman, R. J.; Bao, J.; Pieper, A.; Wang, Z.-Q.; Dawson, T. M.; Snyder, S. H.; Dawson, V. L., Poly(ADP-ribose) polymerase gene disruption renders mice resistant to cerebral ischemia, Nature Med. 3 (1997) 1089-1095.

38. Takahashi, K.; Pieper, A. A.; Croul, S. E.; Zhang, J.; Snyder, S. H.; Greenberg, J. H., Post-treatment with an inhibitor of poly(ADP-ribose) polymerase attenuates cerebral damage in focal ischemia, Brain Res. 829 (1999) 46-54.

39. Banaski, M.; Komura, H.; Shimoyama, M.; Ueda, K., Specific inhibitors of poly(ADP-ribose) synthetase and mono(ADP-ribosyl) transferase, J. Biol. Chem. 267 (1992) 1569-1575.

40. Suto et al. Substituted dihydroisoquinolinones and related compounds as potentiators of the lethal effects of radiation and certain chemotherapeutic agents; selected compounds, analogs and process, U.S. Pat. No. 5,177,075.

41. Moroni, F.; Meli, E.; Peruginelli, F.; Chiarugi, A.; Cozzi, A.; Picca, R.; Romagnoli, P.; Pellicciari, R.; Pellegrini-Giampietro, D. E., Poly(ADP-ribose) polymerase inhibitors attenuate necrotic but not apoptotic neuronal death in experimental models of cerebral ischemia, Cell Death and Diff. 8 (2001) 921-932.

1. A compound having the structure of compound 1:

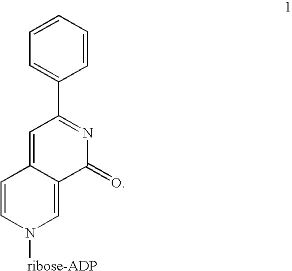

2. A method of preparing the compound of claim 1, comprising:
mixing NAD+ with acetophenone and base, to form a mixture; and
reacting the mixture with acid.

3. The method of claim 2, wherein the reacting comprises adding acid to the mixture and heating.

4. The method of claim 2, wherein the base is a solution of KOH.

5. The method of claim 2, wherein the acid comprises formic acid.

6. A method of detecting NAD+, comprising:
converting NAD+ to a fluorescent compound; and
detecting the fluorescence of the fluorescent compound.

7. The method of claim 6, wherein the fluorescent compound is compound 1:

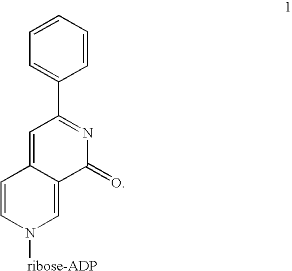

8. The method of claim 6, wherein the converting comprises:
mixing NAD+ with acetophenone and base, to form a mixture; and
reacting the mixture with acid.

9. The method of claim 8, wherein the base is a solution of KOH.

10. The method of claim 8, wherein the acid comprises formic acid.

11. The method of claim 8, wherein the fluorescent compound is compound 1:

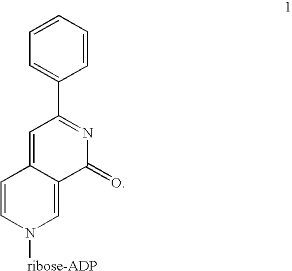

12. A method of quantifying NAD+, comprising:
converting NAD+ to a fluorescent compound; and
measuring an amount of fluorescence of the fluorescent compound.

13. The method of claim 12, wherein the fluorescent compound is compound 1:

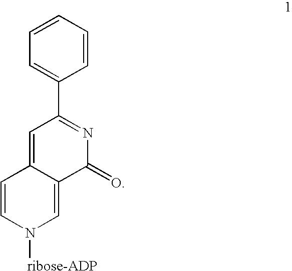

14. The method of claim 12, wherein the converting comprises:
mixing NAD+ with acetophenone and base, to form a mixture; and
reacting the mixture with acid.

15. The method of claim 14, wherein the base is a solution of KOH.

16. The method of claim 14, wherein the acid comprising formic acid.

17. The method of claim 14, wherein the fluorescent compound is compound 1:

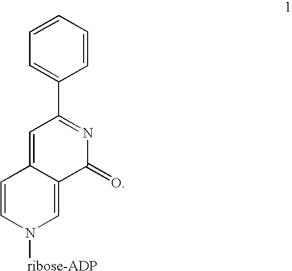

18. A method of detecting an NAD+ utilizing enzyme, comprising:
incubating the enzyme with NAD+ and a substrate for the enzyme;
quantifying any remaining NAD+ by the method of claim 12.

19. The method of claim 18, wherein the fluorescent compound is compound 1:

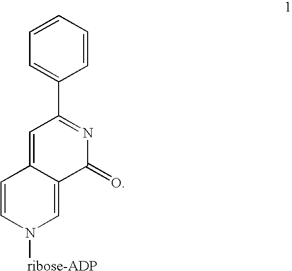

20. The method of claim 18, wherein the converting comprises:
mixing NAD+ with acetophenone and base, to form a mixture; and
reacting the mixture with acid.

21. The method of claim 20, wherein the base is a solution of KOH.

22. The method of claim 20, wherein the acid comprises formic acid.

23. The method of claim 20, wherein the fluorescent compound is compound 1:

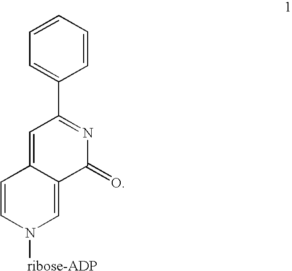

24. The method of claim 18, wherein the enzyme is PARP.

25. A method of determining whether a compound is an inhibitor of an NAD+ utilizing enzyme, comprising:
comparing an amount of NAD+ consumed during reaction of the enzyme with a substrate for the enzyme, with and without the compound;
wherein the amount of NAD+ not consumed is measured by the method of claim 12.

26. The method of claim 25, wherein the fluorescent compound is compound 1:

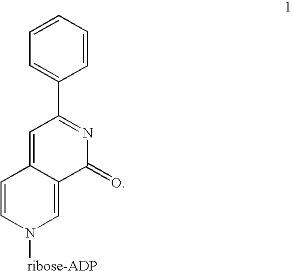

27. The method of claim 25, wherein the converting comprises:
mixing NAD+ with acetophenone and base, to form a mixture; and
reacting the mixture with acid.

28. The method of claim 27, wherein the base is a solution of KOH.

29. The method of claim 27, wherein the acid comprises formic acid.

30. The method of claim 27, wherein the fluorescent compound is compound 1:

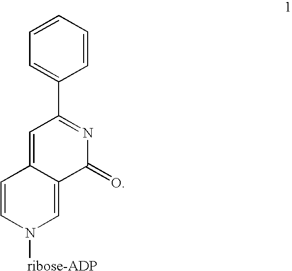

31. The method of claim 25, wherein the enzyme is PARP.

32. The method of claim 27, wherein the enzyme is PARP.

33. A method of detecting a genetic deficiency in an NAD+ utilizing enzyme in a patient, comprising:
comparing an amount of NAD+ consumed during reaction of an enzyme from the patient with a substrate for the enzyme, with an amount of NAD+ consumed during reaction of a control enzyme with the substrate;
wherein the amount of NAD+ not consumed is measured by the method of claim 12.

34. The method of claim 33, wherein the fluorescent compound is compound 1:

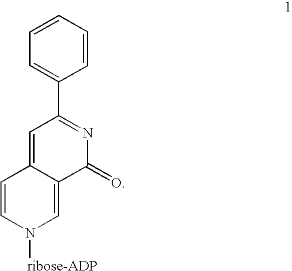

35. The method of claim 33, wherein the converting comprises:
mixing NAD+ with acetophenone and base, to form a mixture; and
reacting the mixture with acid.

36. The method of claim 35, wherein the base is a solution of KOH.

37. The method of claim 35, wherein the acid comprises formic acid.

38. The method of claim 35, wherein the fluorescent compound is compound 1:

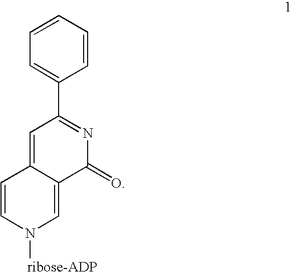

39. The method of claim 33, wherein the NAD+ utilizing enzyme is long-chain 3-hydroxyacyl-CoA dehydrogenase.

40. A kit for detecting NAD+, comprising:
a base,
acetophenone; and
an acid.

41. The kit of claim 40, wherein
the base is a solution of KOH, and
the acid comprises formic acid.

42. A kit of claim 40, further comprising a solution containing a known amount of compound 1:

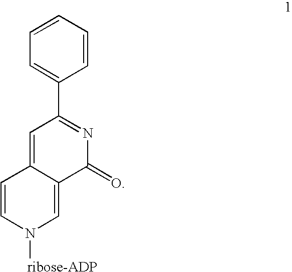

43. A kit of claim 40, further comprising NAD+.

44. A kit for quantifying NAD+, comprising:
a base,
acetophenone;
an acid; and
a standard.

45. A kit of claim 44, wherein the standard is a solution containing a known amount of NAD+.

46. A kit of claim 44, wherein the standard is a solution containing a known amount of compound 1:

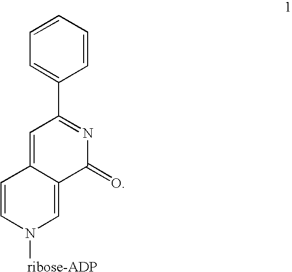

47. The kit of claim 44, wherein
the base is a solution of KOH, and
the acid comprises formic acid.

48. A kit for measuring the activity of an NAD+ utilizing enzyme, comprising:
a base,
acetophenone;
an acid; and
a solution containing a known amount of the NAD+ utilizing enzyme.

49. The kit of claim 48, wherein
the base is a solution of KOH, and
the acid comprises formic acid.

50. A kit of claim 48, further comprising a solution containing a known amount of compound 1:

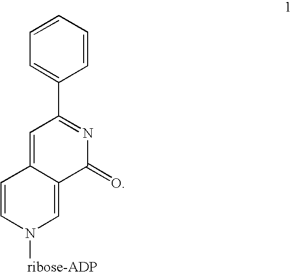

51. A kit of claim 48, further comprising NAD+.

52. A kit of claim 48, wherein the NAD+ utilizing enzyme is PARP.

53. A kit of claim 48, wherein the NAD+ utilizing enzyme is long-chain 3-hydroxyacyl-CoA dehydrogenase.
